(19) United States
(12) Patent Application Publication
(10) Pub. No.: US 2025/0057787 A1
(43) Pub. Date: Feb. 20, 2025

(54) A PHOSPHOLIPID COMPOSITION AND ITS USE IN THE NANO LIQUID PRODUCT CONTAINING CURCUMIN HAVE THE ABILITY TO TREATING BURNS AND INCREASING THE EFFECT OF SCAR HEALING

(71) Applicant: KIM DUNG THI DAO, Ho Chi Minh (VN)

(72) Inventors: KIM DUNG THI DAO, HO CHI MINH (VN); BINH CONG DAO, TAY NINH (VN); TAM CONG NGUYEN, Ho Chi Minh (VN); VAN BICH NGUYEN, Ho Chi Minh (VN); BICH LIEN THI NGUYEN, PHU YEN (VN); THOA THI DAO, HA NOI (VN); NHAT VY DAO NGUYEN, HO CHI MINH (VN)

(73) Assignee: DKD INTERNATIONAL PRODUCTION JOINT STOCK COMPANY, HO CHI MINH (VN)

(21) Appl. No.: 18/451,071

(22) Filed: Aug. 16, 2023

Related U.S. Application Data
(63) Continuation-in-part of application No. 18/450,414, filed on Sep. 6, 2023, now abandoned.

(51) Int. Cl. A61K 31/12; A61K 9/127; A61K 31/355; A61K 31/519; A61K 36/82; A61K 36/886; A61K 47/24; A61K 47/36; A61K 47/46

(52) U.S. Cl. CPC A61K 31/12; A61K 9/127; A61K 9/1277; A61K 31/355; A61K 31/519; A61K 36/82; A61K 36/886; A61K 47/24; A61K 47/36; A61K 47/46

(57) ABSTRACT

A phospholipid composition obtained from the process of forming a homogeneous mixture by mixing (A) a lecithin extract ingredients from egg yolk with (B) a lecithin extract ingredients from plants in a predetermined ratio of (1-3): (1-3); and then adjusting moisture of the homogeneous mixture below 10%. In addition, the invention also discloses the phospholipid composition applied to make the nano liquid product containing curcumin have the ability to treating burns and increasing the effect of scar healing; wherein the product is used at a dose of 0.05-0.1 mL/cm2 of skin, with a frequency of twice daily to reduce the area of the burns, and increasing the concentration of hydroxyproline in the skin.

### CLAIM OF PRIORITY

This application is a continuation application of application Ser. No. 18/450,414 entitled “Nano liquid composition containing curcumin have the ability to treating burns and increasing the effect of scar healing and method of manufacturing the same”, filed on Aug. 16, 2023. The patent application identified above is incorporated here by reference in its entirety to provide continuity of disclosure.

### FIELD OF THE INVENTION

The invention relates to the preparation of lecithin. In particular, the present invention relates to a phospholipid composition and its use in the nano liquid product containing curcumin have the ability to treating burns and increasing the effect of scar healing.

### BACKGROUND ART

NanoLiposome is one of the advanced drug delivery technologies of the nanosystem, which has great significance in the pharmaceutical and cosmetic industries. Now, there have been some drugs prepared from NanoLiposome that have been used in clinical practice with many outstanding advantages compared to conventional drug delivery systems. NanoLiposomes are nano-sized spherical particles consisting of one or more double layers of phospholipid membranes surrounding the core containing the active ingredient. Liposomes are considered an ideal transport system with the ability to store, protect, transport, and release active substances to desired locations in the body. Using NanoLiposome as a drug delivery system in the body is a new direction. NanoLiposome technology is applied to anti-cancer drugs, gene fragments, recombinant proteins, etc., based on the pharmacokinetic characteristics of NanoLiposome active substances will be transported to target cells precisely, helping to improve the effective treatment and reducing toxicity compared to traditional drugs. Liposome technology also helps poorly soluble active ingredients penetrate into the small intestine wall, helping the body absorb quickly and increase the effectiveness of treatment.

Lecithin is a phospholipid involved in the composition of cell membranes produced in the liver. Lecithin increases the absorption of food and is a good soluble medium for vitamins A, D, E, and K. Lecithin is found naturally in the body's tissues. It is made up of fatty acids and has many commercial and medical uses. Lecithin is used as an emulsifier in the food, pharmaceutical and cosmetic industries.

Enzyme lipase, also known as tryaciglycerol lipase, is an enzyme capable of catalyzing the hydrolysis of long-chain triacylglycerols to diacylglycerol, monoacylglycerol, glycerol, and free fatty acids at the interface between water and organic solvents. In addition, lipase also participates in catalyzing ester translocation reactions and ester synthesis reactions in a low-water environment. Belongs to the group of hydrolase enzymes, specifically cutting ester bonds. Strongly active in emulsifying systems, especially island emulsion systems. At the interface between the aqueous phase and the insoluble phases containing the substrate.

According to Patent No. RU2058787C1, the invention refers to the lecithin preparation method that includes the following steps: hen egg yolks were homogenized in acetone at a temperature from-20 to-25 C for 3 min. The process is repeated 6 times. Then egg yolk is extracted with ethanol at 24-28 C in an inert gas atmosphere for 1.5 hr. Obtained after filtration, the cleared solution is precipitated with cadmium chloride. Precipitate is reprecipitated 5 times with ethanol containing cadmium chloride, dissolved in chloroform, and treated with 30% ethanol solution.

According to Patent No. U.S. Pat. No. 4,157,404A, the invention refers to a process for obtaining yolk lecithin from a raw egg yolk which comprises subjecting a raw egg yolk to extraction with liquid dimethyl ether to obtain an extract and dehydrating the extract to the extent that the water content is not more than 20% by weight, whereby a lecithin-rich fraction is obtained as a separate phase from a neutral lipids-fraction. The isolated lecithin-rich fraction may further be subjected to second-stage dehydration to give a product in which the lecithin content is as high as 50 to 85% by weight.

Chakraborty, D., et al. (in Evaluation of the parameters affecting the extraction of sesame oil from sesame (Sesamum indicum L.) seed using soxhlet apparatus) International Food Research Journal 24 (2): 691-695 (April 2017) describe the solvent extraction method was carried out using soxhlet apparatus to extract sesame oil from the sesame seed. The primary processing of sesame seed before oil extraction includes drying, crushing, and separating.

According to Patent No. U.S. Pat. No. 5,955,327, the invention refers to a process for manufacturing vegetable lysolecithins from hydrated lecithin used as a starting material which comprises allowing a hydrolysis enzyme consisting of phospholipase A1 or A2 to act on hydrated lecithin, then adding to the resultant lysolecithin solution acetone in a proportion on a volume basis of either 1 to 4, or at least 5, compare to the water contained in said resultant lysolecithin solution to thereby either float or precipitate the lysolecithin phase, then separating out said lysolecithin phase, followed by the repetition of the acetone extraction procedure to separate for removal free fatty acids by-produced in the said lysolecithin production reaction as well as oil-soluble impurities originated from the starting material.

According to Patent No. CN114532535, the invention refers to the preparation method of curcumin nano-liposome comprises the following steps: (1) weighing soybean lecithin, cholesterol, and curcumin, dissolving in an organic solvent, performing rotary evaporation, and removing the organic solvent to form a film; (2) hydrating the lipid film with phosphate buffer solution to form coarse liposome, and stirring; and (3) standing after ultrasonic treatment, and filtering to obtain the nano liposome.

The above inventions meet the specific purposes and requirements of a technical solution. However, the disclosure of the invention does not in detail the process of lipase enzyme recovery obtained from germinated dragon fruit seeds to break down large molecule proteins into small molecule peptides, speeding up the separation of lecithin and proteins; undisclosed the cellulose enzyme system and the pectinase enzyme were obtained from any microorganism source, as well as the combined germination stimulation method between the enzyme system and GA3, and the technical parameters were also different; besides, specifications related to concentration, time, temperature and order of use in combination of extraction, separation, and purification methods are also different; Liposome preparations containing lecithin extract as emulsifier also differed from the control in lecithin extract composition.

Therefore, it is necessary to create a process of recalling the lipase enzyme obtained from germinated dragon fruit seeds to break down large molecule proteins into small molecule peptides, speeding up the separation of lecithin and proteins.

It is necessary to create a process of making lecithin extract ingredients from egg yolk raw materials and plant materials.

Finally, It is necessary to provide a nano liposomal preparation containing the extract lecithin as an emulsifier.

The invention provides solutions to achieve the above objectives.

### SUMMARY OF THE INVENTION

Accordingly, an objective of the present invention is to provide a phospholipid composition obtained from the process of forming a homogeneous mixture by mixing (A) a lecithin extract ingredients from egg yolk with (B) a lecithin extract ingredients from plants in a predetermined ratio of (1-3): (1-3); and then adjusting moisture of the homogeneous mixture below 10%;

wherein (A) the lecithin extract ingredients from egg yolk is obtained by performed in a specific order from (a) to (g) comprising:
(a) selecting and preparing an egg yolks by predetermined quality guidelines, then treating egg yolks by washed with water, and using the sieve to remove membranes of egg yolks;
(b) hydrolyzing said treated egg yolks with a lipase enzyme preparation at 35° C. for 2 hours to create an enzyme-treated egg yolks; wherein the lipase enzyme preparation having 0.05%-1.5% by weight of the said treated egg yolks;
(c) adding and homogeneously dissolving acetone solution to the enzyme-treated egg yolks for 5-10 minutes, then let stand at 3° C.-5° C. for 2-3 hours for precipitate phospholipids and proteins;
(d) creating a first precipitate by performing the steps in the following order:
   (d1) filtering said phospholipid and protein precipitate generated at step (c);
   (d2) washing said precipitate at step (d1) with acetone solution, and then filtering; repeating steps of at step (d2) 4 times;
   (d3) removing acetone from said precipitate at step (d2) to create the first precipitate;
(e) creating a second precipitate by adding and homogeneously dissolving 5 parts of ethanol solution with 1 part of the first precipitate at 2° C.-5° C. for 5-8 minutes, and then filtering;
(f) creating a basic mixture by performing the steps in the following order:
   adding and homogeneously dissolving 5 parts of ethanol 91% with 1 part of the second precipitate, and admixing a powder of activated carbon; then let stand for 15 min;
   performing extraction and removing ethanol by rotary evaporator to obtain the basic mixture;
   wherein the powder of activated carbon having 0.005% by total weight of ethanol 91% and the second precipitate; and
(g) creating the lecithin extract ingredients from egg yolk by performing the steps in the following order:
   adding and homogeneously dissolving 3 parts of petroleum ether with 1 part of the basic mixture and 1 part of acetone, then let stand at 2° C.-5° C. for 1-3 hours;
   removing the solvent layer on the surface, twice extracting by chloroform, and removing the solvent at low pressure to obtain the lecithin extract ingredients from egg yolk;
      wherein (B) the lecithin extract ingredients from plants is obtained by performed in a specific order from (a′) to (g′) comprising:
(a′) creating a first mixture by performing the steps in the following order: chopping/cutting 1 part of an avocado by-product and 3 parts of an eggplant (Solanum melongena) to obtain a temporary mixture;
   treating the temporary mixture by steam at 100° C. for 5 minutes to obtain a steam-treated temporary mixture;
   grinding steam-treated temporary mixture to obtain a temporary powder; and
   drying the temporary powder having moisture content below 7% to obtain the first mixture;
(b′) creating a second mixture by performing the steps in the following order:
   admixing 1 part of a tomato seeds (Solanum lycopersicum) with 1 part of a chili seeds and 2 parts of a sesame seeds (Sesamum indicum) to obtain a seeds mixture;
   treating the seeds mixture by steam at 100° C. for 5 minutes, then grinding to obtain a seeds powder;
   treating the seeds powder by cellulose enzyme ingredient at 28° C.-35° C. for 1 hour to obtain an enzyme-treated seeds powder; wherein cellulose enzyme ingredient having 0.05%-0.5% by weight of the seeds powder;
   drying the enzyme-treated seeds powder having moisture content below 7% to obtain the second mixture;
(c′) adding into the soxhlet extractor consisting of 1 part of the first mixture with 1 part of the second mixture, 1 part of a rice bran having moisture content less than 7%, and (10-15) parts of ethyl ether solvent; all proceed to extract for 8 hours to obtain a temporary solution and a residue;
(d′) creating a first solution by centrifugation of the temporary solution;
(e′) creating a second solution by treated the residue with the lipase enzyme preparation at 35° C. for 2 hours; wherein the lipase enzyme preparation having 0.05%-1.5% by weight of the residue;
centrifuging said enzyme-treated residue and removing solvents by vacuum evaporator at 40° C. to obtain the second solution;
(f′) creating a foundation solution by adding and homogeneously dissolving the first solution with the second solution, and the powder of activated carbon, then let stand for 15 minutes, and centrifuging; wherein the powder of activated carbon having 0.005% by total weight of the first solution and the second solution; and
(g′) creating the lecithin extract ingredients from plants by performing the steps in the following order:
   (g1′) adding and homogeneously dissolving cold acetone (4° C.) to the foundation solution for 5 minutes, then let stand for 3 hours at 5° C. for precipitate lecithin;
   (g2′) washing said lecithin precipitate at step (g1′) by acetone at room temperature, and then filtering; repeating steps of step (g2′) 5 times;
   (g3′) adding and homogeneously dissolving cold ethanol with said precipitate at step (g2′) for 8-10 minutes; and
   (g4′) filtering and removing solvents by vacuum evaporator at 40° C., then twice extracting by chloroform, and removing the solvent at low pressure to obtain the lecithin extract ingredients from plants.

Another objective of the present invention is to provide a lipase enzyme preparation obtained from a germinated dragon fruit seeds include the following steps:
(i) preparing the germinated dragon fruit seeds by following these steps in order:
   selecting and preparing a dragon fruits by predetermined quality guidelines;
   washing, peeling, cutting/chopping said dragon fruits into pieces and soaking with an enzyme solution according a ratio 1:5 at 30° C.-35° C. for 12 hours;
      wherein the enzyme solution is obtained by homogeneously dissolving 1 part of an enzyme preparation with 1000 parts of water;
      wherein the enzyme preparation including 2 parts of cellulase enzyme and 3 parts of pectinase enzyme;
      wherein said cellulase enzyme is the biomass obtained by the process of increasing the biomass of microbial strains including Bacillus lichenifomis DA 15, Bacillus subtilis DA 33, and Bacillus megaterium NT09;
      wherein said pectinase enzyme is the biomass obtained by the process of increasing the biomass of microbial strains including Aspergillus niger X5, and Aspergillus niger X9;
   treating said enzyme-treated dragon fruits by steam at the temperature of 90° C. for 3 minutes;
   scrubbing said steam-treated dragon fruits by multipurpose scrubber separating a part of pure, and a part of seeds;
   soaking the part of seeds with GA3 solution at a ratio of 1:10 for 6 hours at 30° C.-35° C.; wherein GA3 solution is obtained by homogeneously dissolving 0.5 mg of GA3 with 1L of water;
   incubating said GA3-treated part of seeds at 30° C.-35° C. for 8-12 hours, and maintain humidity of 75%-80% to obtain the germinated dragon fruit seeds;
(ii) creating a solution after centrifugation by following these steps in order:
   grinding a mixture consisting of ½ part of water and 1 part of the germinated dragon fruit seeds;
   stirring said after grinding mixture for 30 minutes;
   centrifuging said after stirring mixture at 13000 rpm for 10 minutes at 4° C. to obtain a solution after centrifugation; and
   freeze-drying the solution after centrifugation to obtain the lipase enzyme preparation form powder, that having activity of 12-16 TU/mg powder.

Yet another objective of the present invention is to provide a phospholipid composition applied to make the nano liquid product containing curcumin have the ability to treating burns and increasing the effect of scar healing, wherein the nano liquid product containing curcumin obtained by homogenously mixing a curcumin nano ingredient with the phospholipid composition in a ratio of 1:1 for 4 hours at 120° C. by emulsifying equipment;
wherein the curcumin nano ingredient is obtained by performed in a specific order from (i) to (v) comprising:
   (i) preparing a dispersed phase by dissolving 4 parts of a curcumin with 5 parts of ethanol;
   (ii) creating a carrier mixture by homogeneous dissolving 1.5 parts PEG (polyethylene glycol) with 6 parts EG (ethylene glycol) and 2 parts water, with the combination of ultrasonic vibration for 2 hours at room temperature;
   (iii) creating a homogeneous mixture by homogeneous dissolving 1.6 parts of the dispersed phase at step (i) with 1.5 parts of the carrier mixture at step (ii), and 2 parts of the phospholipid composition by emulsifying equipment;
   (iv) keeping the homogeneous mixture overnight; and
   (v) centrifuging said homogenized mixture at step (iv) with a speed of 5000 rpm for 10 min, and repeated 6 times of centrifugation to obtain the curcumin nano ingredient form microemulsion;
      wherein the product is used at a dose of 0.05-0.1 mL/cm2 of skin, with a frequency of twice daily to reduce the area of the burns, and increasing the concentration of hydroxyproline in the skin.

Finally, the purpose of the invention is to provide a phospholipid composition applied to make the nano liquid product containing curcumin have the ability to treating burns and increasing the effect of scar healing, wherein the nano liquid product containing curcumin obtained by homogenously mixing a curcumin nano ingredient with a foundation mixture in a ratio of 1:1 for 4 hours at 120° C. by emulsifying equipment; wherein the foundation mixture comprising cholesterol, folic acid, nanocurumin, tocopherol, xanthan gum, and the phospholipid composition.

These and other advantages of the present invention will no doubt become obvious to those of ordinary skill in the art after having read the following detailed description of the preferred embodiments, which are illustrated in the various drawing Figures.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 is a conceptual block diagram illustrating the principle of making the phospholipid composition in accordance with an exemplary embodiment of the present invention; and

FIG. 2 is a flowchart illustrating a flowchart of a general method of manufacturing the lipase enzyme preparation from a germinated dragon fruit seeds in accordance with an exemplary embodiment of the present invention.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well-known methods, procedures, components, and circuits have not been described in detail so as not to unnecessarily obscure aspects of the present invention.

In the embodiment of the present invention, percent mass or percentage (%) by weight=(mass of solute/mass of solution)×100%. The unit of mass is usually grams. Mass percent is also known as the correct percentage by weight or w/w %. It should also be noted that the molar mass is also within the meaning of the invention. Molar mass is the total mass of all atoms in a mole of compound. Total all volume percentages add up to 100%.

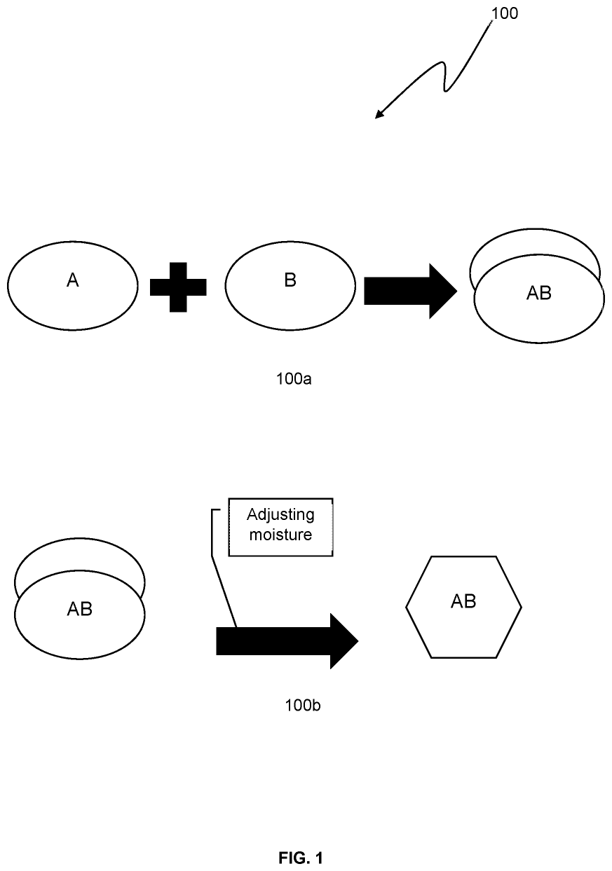

One embodiment of the invention is now described with reference to FIG. 1. FIG. 1 illustrates a conceptual block diagram of a method 100 including a first stage 100a and a second stage 100b of manufacturing the phospholipid composition in accordance with an exemplary embodiment of the present invention.

In the first stage of 100a, a homogeneous mixture by mixing a lecithin extract ingredients from egg yolk (A) with a lecithin extract ingredients from plants (B) in a predetermined ratio of (1-3): (1-3). In many aspects of the present invention, the homogeneous solution is defined as a mixture with the following functions including an emollient (think softening), water-binding agent (to boost hydration), and emulsifying agent (to help water and oil ingredients mesh better in a formula).

In the second stage of 100b, adjusting moisture of the homogeneous mixture below 10% to create a phospholipid composition (AB).

According to the preferred embodiment of the invention, the phospholipid composition (AB) applied to make the nano liquid product containing curcumin have the ability to treating burns and increasing the effect of scar healing; wherein the product is used at a dose of 0.05-0.1 mL/cm2 of skin, with a frequency of twice daily to reduce the area of the burns, and increasing the concentration of hydroxyproline in the skin.

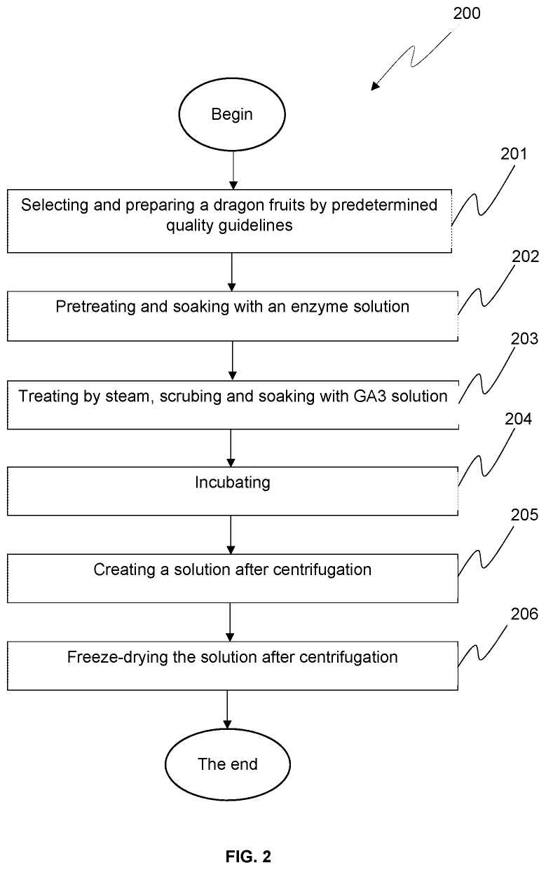

According to the preferred embodiment of the invention, the lecithin extract ingredients from egg yolk (A) is obtained by performed in a specific order from (a) to (g) comprising:
(a) selecting and preparing an egg yolks by predetermined quality guidelines, then treating egg yolks by washed with water, and using the sieve to remove membranes of egg yolks; wherein, the egg yolk is selected from eggs of non-infected poultry, meeting the parameters including: sensory parameters (state, color, and taste); digestive-physical indicators (moisture, protein, carbohydrates, and energy); microbiological parameters (total aerobic microorganisms, Colifroms, E. coli, S. aureus, Cl. Perfringens, Streptococci faecal, Pseidomonas aeruginosa, Baccilus cereus, and Salmonella); and metal indicators (cadmium, arsenic, lead, and mercury);
(b) hydrolyzing said treated egg yolks with a lipase enzyme preparation at 35° C. for 2 hours to create an enzyme-treated egg yolks; wherein the lipase enzyme preparation having 0.05%-1.5% by weight of the said treated egg yolks; and the lipase enzyme preparation obtained from a process 200 (referred to by FIG. 2) will be described later;
(c) adding and homogeneously dissolving acetone solution to the enzyme-treated egg yolks for 5-10 minutes, then let stand at 3° C.-5° C. for 2-3 hours for precipitate phospholipids and proteins;
(d) creating a first precipitate by performing the steps in the following order:
   (d1) filtering said phospholipid and protein precipitate generated at step (c);
   (d2) washing said precipitate at step (d1) with acetone solution, and then filtering; repeating steps of at step (d2) 4 times;
   (d3) removing acetone from said precipitate at step (d2) to create the first precipitate;
(e) creating a second precipitate by adding and homogeneously dissolving 5 parts of ethanol solution with 1 part of the first precipitate at 2° C.-5° C. for 5-8 minutes, and then filtering;
(f) creating a basic mixture by performing the steps in the following order:
   adding and homogeneously dissolving 5 parts of ethanol 91% with 1 part of the second precipitate, and admixing a powder of activated carbon; then let stand for 15 min;
   performing extraction and removing ethanol by rotary evaporator to obtain the basic mixture;
   wherein the powder of activated carbon having 0.005% by total weight of ethanol 91% and the second precipitate; and
(g) creating the lecithin extract ingredients from egg yolk by performing the steps in the following order:
   adding and homogeneously dissolving 3 parts of petroleum ether with 1 part of the basic mixture and 1 part of acetone, then let stand at 2° C.-5° C. for 1-3 hours;
   removing the solvent layer on the surface, twice extracting by chloroform, and removing the solvent at low pressure to obtain the lecithin extract ingredients from egg yolk (A).

According to the preferred embodiment of the invention, the lecithin extract ingredients from egg yolk (A) are selected from eggs of various types of poultry including chicken (Gallus gallus domesticus, Gallus gallus otomesticus), red-feathered wild chicken (Gallus gallus spadiceus), muskuseend (Cairina moschata forma domestica), grass goose (Cynopsis sinensis), mallard (Anas platyrhynchos), and quail (Coturnix japonica); all are listed in detail in Table 1 below.

TABLE 1
Scientific name and distribution of types poultry used for egg
yolk harvesting as embodiment of the present invention.

| No. | Name | Science name | Distribution |
|---|---|---|---|
| 1 | Chicken | Gallus gallus domesticus | Viet Nam |
| 2 | Red-feathered | Gallus gallus spadiceus | Northwest |
| | wild chicken | | Viet Nam |
| 3 | Chicken | Gallus gallus tomesticus | Bac Giang, Hai |
| | | | Phong, Hai Duong . . . |
| 4 | Muskuseend | Cairina moschata forma | Red river delta |
| | | domestica | |
| 5 | Grass goose | Cynopsis sinensis | Northern Plains and |
| | | | Midlands |
| 6 | Mallard | Anas platyrhynchos | Ben Tre, Nghe An |
| 7 | Quail | Coturnix japonica | Dong Nai |

According to the preferred embodiment of the invention, the lecithin extract ingredients from plants (B) comprising an avocado by-product, an eggplant (Solanum melongena), a tomato seeds (Solanum lycopersicum), a sesame seeds (Sesamum indicum), and a rice bran; all are listed in detail in Table 2 below.

TABLE 2
Scientific names and distribution of plant species used for
lecithin extract as embodiment of the present invention.

| No. | Name | Science name | Distribution |
|---|---|---|---|
| 1 | Avocado 034 | Persea americana | Bao Loc-Lam Dong |
| 2 | Avocado wax | Persea americana | Dak Lak |
| | | ‘Maluma’ | |
| 3 | Eggplant | Solanum melongena | Dak Nong |
| 4 | Sesame seeds | Sesamum indicum | Thanh Hoa, Nghe |
| | | | An, Ha Tinh, |
| | | | Quang Binh, Quang Tri |
| 5 | Rice bran | Rice bran | Mekong delta |
| 6 | Celestial chili | Celestial chili | Thanh Binh, Dong Thap |
| 7 | Dangerous chili | Capsicumn annuum L | Thanh Binh, Dong Thap |
| 8 | Ba tri chili | Golden Horn Peppers | Ben Tre |
| 9 | Tomato | Solanum lycopersicum | Lam Dong |

According to the embodiment of the invention, the lecithin extract ingredients from plants (B) is obtained by performed in a specific order from (a′) to (g′) comprising:
(a′) creating a first mixture by performing the steps in the following order:
   chopping/cutting 1 part of an avocado by-product and 3 parts of an eggplant (Solanum melongena) to obtain a temporary mixture;
   treating the temporary mixture by steam at 100° C. for 5 minutes to obtain a steam-treated temporary mixture;
   grinding steam-treated temporary mixture to obtain a temporary powder; and
   drying the temporary powder having moisture content below 7% to obtain the first mixture;
   wherein the avocado by-product include skins and seeds selected from types of avocado including avocado 034 (Persea americana), and avocado wax (Persea americana ‘Maluma’);
(b′) creating a second mixture by performing the steps in the following order:
   admixing 1 part of a tomato seeds (Solanum lycopersicum) with 1 part of a chili seeds and 2 parts of a sesame seeds (Sesamum indicum) to obtain a seeds mixture;
   treating the seeds mixture by steam at 100° C. for 5 minutes, then grinding to obtain a seeds powder;
   treating the seeds powder by cellulose enzyme ingredient at 28° C.-35° C. for 1 hour to obtain an enzyme-treated seeds powder; wherein cellulose enzyme ingredient having 0.05%-0.5% by weight of the seeds powder;
   drying the enzyme-treated seeds powder having moisture content below 7% to obtain the second mixture;
   wherein the chili seeds are selected from types of chili including celestial chili (Celestial chili), Dangerous chili (Capsicumn annuum L.), and Ba tri chili (Golden Horn Peppers);
(c′) adding into the soxhlet extractor consisting of 1 part of the first mixture with 1 part of the second mixture, 1 part of a rice bran having moisture content less than 7%, and (10-15) parts of ethyl ether solvent; all proceed to extract for 8 hours to obtain a temporary solution and a residue;
(d′) creating a first solution by centrifugation of the temporary solution;
(e′) creating a second solution by treated the residue with the lipase enzyme preparation at 35° C. for 2 hours; and centrifuging said enzyme-treated residue and removing solvents by vacuum evaporator at 40° C. to obtain the second solution;
   wherein the lipase enzyme preparation having 0.05%-1.5% by weight of the residue; and the lipase enzyme preparation obtained from the process 200 (referred to by FIG. 2) will be described later;
(f′) creating a foundation solution by adding and homogeneously dissolving the first solution with the second solution, and the powder of activated carbon, then let stand for 15 minutes, and centrifuging; wherein the powder of activated carbon having 0.005% by total weight of the first solution and the second solution; and
(g′) creating the lecithin extract ingredients from plants by performing the steps in the following order:
   (g1′) adding and homogeneously dissolving cold acetone (4° C.) to the foundation solution for 5 minutes, then let stand for 3 hours at 5° C. for precipitate lecithin;
   (g2′) washing said lecithin precipitate at step (g1′) by acetone at room temperature, and then filtering; repeating steps of step (g2′) 5 times;
   (g3′) adding and homogeneously dissolving cold ethanol with said precipitate at step (g2′) for 8-10 minutes; and
   (g4′) filtering and removing solvents by vacuum evaporator at 40° C., then twice extracting by chloroform, and removing the solvent at low pressure to obtain the lecithin extract ingredients from plants (B).

It should be noted that these apparatus used according to the method 100 in the present invention includes, but is not limited to a centrifuge, a crusher, a filtration equipment, a cutting machines, an emulsifying equipment, a washing machine, a stirring equipment, a multipurpose scrubber, a scrubbers, and others similar devices, that all have been known in previous art so the description of the structure and its operating principle will not be described in detail in the invention.

Now referring to FIG. 2, the process 200 is the manufacturing the lipase enzyme preparation from a germinated dragon fruit seeds in accordance with embodiment of the present invention. Process 200 starts with step 201 which is to select and prepare dragon fruits by predetermined quality guidelines, that include selecting the dragon fruits have a Brix level of at least 10; and performing visual inspection to select said fruits that are ripe, undamaged, heavy, and free of spoilage spots.

According to the preferred embodiment of the invention, the dragon fruits are selected from types of dragon fruits including red flesh dragon fruit (Hylocereus costaricensis), white flesh dragon fruit (Selenicereus undatus), Gele pitahaya (Hylocereus megalanthus), and Purple and pink dragon fruit (Hylocereus undatus costaricensis); all listed in detail in Table 3 below.

TABLE 3
Scientific names and distribution of types of dragon fruits used
for dragon fruit seeds harvesting as embodiment of the present invention.

| No. | Name | Science name | Distribution |
|---|---|---|---|
| 1 | Red flesh dragon fruit | Hylocereus | Binh Thuan, Long An, |
| | | costaricensis | Tien Giang |
| 2 | White flesh dragon fruit | Selenicereus | Binh Thuan, Long An, |
| | | undatus | Tien Giang |
| 3 | Gele pitahaya | Hylocereus | Binh Thuan, Long An, |
| | | megalanthus | Tien Giang |
| 4 | Purple and pink dragon | Hylocereus undatus | Binh Thuan, Long An, |
| | fruit | costaricensis | Tien Giang |

Continuing with FIG. 2, at step 202, pretreating including washing, peeling, cutting/chopping the dragon fruits at step 201 into pieces. Then, soaking said pieces with an enzyme solution according a ratio 1:5 at 30° C.-35° C. for 12 hours. The result in step 202 obtained an enzyme-treated dragon fruits.

According to the embodiment of the invention, the enzyme solution is obtained by homogeneously dissolving 1 part of an enzyme preparation with 1000 parts of water; wherein the enzyme preparation including 2 parts of cellulase enzyme and 3 parts of pectinase enzyme.

According to the preferred embodiment of the invention, cellulase enzyme are obtained froming a process proliferation of microbial strains including Bacillus lichenifomis DA 15, Bacillus subtilis DA 33, and Bacillus megaterium NT09; all are listed in Table 4 below.

According to the preferred embodiment of the invention, pectinase enzyme are obtained froming a process proliferation of microbial strains including Aspergillus niger X5, and Aspergillus niger X9; all are listed in Table 4 below.

TABLE 4
Microbial strains used for producing cellulase enzyme and pectinase
enzyme according to the embodiment of the present invention.

| Features | Name of | References |
|---|---|---|
| Cellulase | Bacillus lichenifomis | Trang NT, and Ha NTH, 2015. |
| | ÐA 15; | The grown and synthetic |
| enzyme | Bacillus subtilis ÐA 33; | capacity of cellulase by |
| | Bacillus megaterium | some Bacillus strains isolated in |
| | NT09. | VietNam. The 6th national |
| | | conference on ecology |
| | | and biological resources. |
| Pectinase | Aspergillus niger X5; | Dung TQ, Cuc NT, Suong |
| enzyme | Aspergillus niger X9. | NT, and Chung |
| | | NTT, 2015. Isolation and |
| | | screening of Aspergillus |
| | | niger strains for biosynthesis |
| | | of pectianse from peels of |
| | | some fruits, (banana, |
| | | apple, mango, dragon and |
| | | carrot) in Hue city. The 6th |
| | | national conference on ecology |
| | | and biological resources. |

At step 203, creating to a GA3-treated part of seeds by treating by steam, scrubbing the enzyme-treated dragon fruits at step 202, then soaking with a GA3 solution. According to the embodiment of the invention, a steam-treated dragon fruits obtained by treating the enzyme-treated dragon fruits at step 202 by steam at the temperature of 90° C. for 3 minutes. Then, scrubbing said steam-treated dragon fruits by multipurpose scrubber separating a part of pure, and a part of seeds. The part of seeds is soaked with the GA3 solution at a ratio of 1:10 for 6 hours at 30° C.-35° C. According to the embodiment of the invention, the GA3 solution is obtained by homogeneously dissolving 0.5 mg of GA3 with 1L of water.

At step 204, incubating the GA3-treated part of seeds at 30° C.-35° C. for 8-12 hours, and maintain humidity of 75%-80% to obtain the germinated dragon fruit seeds.

At step 205, creating a solution after centrifugation by following these steps in order:
grinding a mixture consisting of ½ part of water and 1 part of the germinated dragon fruit seeds at step 204;
stirring said after grinding mixture for 30 minutes; and
centrifuging said after stirring mixture at 13000 rpm for 10 minutes at 4° C. to obtain the solution after centrifugation.

Finally, at step 206, freeze-drying the solution after centrifugation to obtain the lipase enzyme preparation form powder, that having activity of 12-16 TU/mg powder.

According to the embodiment of the invention, the phospholipid composition applied to make the nano liquid product containing curcumin have the ability to treating burns and increasing the effect of scar healing; wherein the nano liquid product containing curcumin have the ability to treating burns and increasing the effect of scar healing 300 (“product 300”) is used at a dose of 0.05-0.1 mL/cm2 of skin, with a frequency of twice daily to reduce the area of the burns, and increasing the concentration of hydroxyproline in the skin.

According to the preferred embodiment of the invention, the product 300 obtained by homogenously mixing a curcumin nano ingredient with the phospholipid composition in a ratio of 1:1 for 4 hours at 120° C. by emulsifying equipment; wherein the phospholipid composition is created according to the method 100 described in detail above.

According to the embodiment of the invention, the product 300 containing the curcumin nano ingredient is obtained by performed in a specific order from (i) to (v) comprising:
(i) preparing a dispersed phase by dissolving 4 parts of a curcumin with 5 parts of ethanol;
(ii) creating a carrier mixture by homogeneous dissolving 1.5 parts PEG (polyethylene glycol) with 6 parts EG (ethylene glycol) and 2 parts water, with the combination of ultrasonic vibration for 2 hours at room temperature;
(iii) creating a homogeneous mixture by homogeneous dissolving 1.6 parts of the dispersed phase at step (i) with 1.5 parts of the carrier mixture at step (ii), and 2 parts of the phospholipid composition by emulsifying equipment;
(iv) keeping the homogeneous mixture overnight; and
(v) centrifuging said homogenized mixture at step (iv) with a speed of 5000 rpm for 10 min, and repeated 6 times of centrifugation to obtain the curcumin nano ingredient form microemulsion.

According to the another embodiment of the invention, the phospholipid composition applied to make the nano liquid product containing curcumin have the ability to treating burns and increasing the effect of scar healing; wherein the nano liquid product containing curcumin have the ability to treating burns and increasing the effect of scar healing 400 (“product 400”) is used at a dose of 0.05-0.1 mL/cm2 of skin, with a frequency of twice daily to reduce the area of the burns, and increasing the concentration of hydroxyproline in the skin.

According to the preferred embodiment of the invention, the product 400 obtained by homogenously mixing a curcumin nano ingredient with a foundation mixture in a ratio of 1:1 for 4 hours at 120° C. by emulsifying equipment.

According to the embodiment of the invention, the foundation mixture comprising cholesterol, folic acid, nanocurumin, tocopherol, xanthan gum, and the phospholipid composition; wherein the phospholipid composition is created according to the method 100 described in detail above.

According to the embodiment of the invention, the foundation mixture comprising: cholesterol having 18%-25% by weight; folic acid having 8%-12% by weight; nanocurumin having 12%-20% by weight; tocopherol having 2%-5% by weight; xanthan gum having 2%-5% by weight; Camellia Sinensis extracts ingredient having 2%-5% by weight; aloe vera extracts ingredient having 0.25%-1% by weight; and the remainder is the phospholipid composition.

According to the preferred embodiment of the invention, the foundation mixture comprising: cholesterol having 21% by weight; folic acid having 9.5% by weight; nanocurumin having 15% by weight; tocopherol having 3% by weight; xanthan gum having 3% by weight; Camellia Sinensis extracts ingredient having 3% by weight; aloe vera extracts ingredient having 0.5% by weight; and the remainder is the phospholipid composition.

According to the embodiment of the invention, the product 400 containing the curcumin nano ingredient is obtained by performed in a specific order from (i) to (v) comprising:
(i) preparing a dispersed phase by dissolving 4 parts of a curcumin with 5 parts of ethanol;
(ii) creating a carrier mixture by homogeneous dissolving 1.5 parts PEG (polyethylene glycol) with 6 parts EG (ethylene glycol) and 2 parts water, with the combination of ultrasonic vibration for 2 hours at room temperature;
(iii) creating a homogeneous mixture by homogeneous dissolving 1.6 parts of the dispersed phase at step (i) with 1.5 parts of the carrier mixture at step (ii), and 2 parts of the phospholipid composition by emulsifying equipment;
(iv) keeping the homogeneous mixture overnight; and
(v) centrifuging said homogenized mixture at step (iv) with a speed of 5000 rpm for 10 min, and repeated 6 times of centrifugation to obtain the curcumin nano ingredient form microemulsion.

It should be noted that the term “admixed/mixed/admixing/mixing” as used in the present invention is understood to mean adding, or reacting, or dissolving homogeneously, or evenly, components in the same solution/mixture. The term “homogeneous/homogeneously” is understood to mean the uniform distribution, or complete dissolution of substances present in a solution/mixture/formula.

According to an embodiment taken as an example of the invention, manufacturing 600 Kg of the phospholipid composition by the method 100, including 15 formulas, listed in detail in Table 5 below.

TABLE 5
Mixed ingredients used for manufacturing the
phospholipid composition including 15 formulas
according to the embodiment of the present invention.

| | Mixed ingredients (Kg) | |
|---|---|---|
| | The lecithin extract | The lecithin extract |
| | ingredients | ingredients |
| Formula | from egg yolk | from plants |
| 1 | 150 | 450 |
| 2 | 200 | 400 |
| 3 | 225 | 375 |
| 4 | 240 | 360 |
| 5 | 257 | 343 |
| 6 | 267 | 333 |
| 7 | 273 | 327 |
| 8 | 300 | 300 |
| 9 | 327 | 273 |
| 10 | 333 | 267 |
| 11 | 343 | 257 |
| 12 | 360 | 240 |
| 13 | 375 | 225 |
| 14 | 400 | 200 |
| 15 | 450 | 150 |

According to the embodiment of the present invention, investigation of three characteristics of the phospholipid composition including emollient ability, water-binding ability, and emulsifying ability of formulations from 1 to 15. Resulting, formulas 1 to 15 will have said three characteristics ascending in order from 1 to 15. Therefore, the phospholipid composition according to the formula from 1 to 15, applied to make 15 the nano liquid products containing curcumin corresponding; wherein efficiency of treating burns and increasing the effect of scar healing of 15 the nano liquid products will increase gradually in order from 1 to 15.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, element components, and/or groups thereof.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application and to enable others of ordinary skill in the art to understand the invention for various embodiments with various modifications as are suited to the particular use contemplated.

The flow diagrams depicted herein are just one example. There may be many variations to this diagram or the steps (or operations) described therein without departing from the spirit of the invention. For instance, the steps may be performed in a differing order, or steps may be added, deleted, or modified. All of these variations are considered a part of the claimed invention.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A phospholipid composition obtained from the process of forming a homogeneous mixture by mixing (A) a lecithin extract ingredients from egg yolk with (B) a lecithin extract mixture in a predetermined ratio of (1-3): (1-3); and then adjusting moisture of the homogeneous mixture;
wherein (A) the lecithin extract ingredients from egg yolk is obtained by performed in a specific order from (a) to (g) comprising:
(a) selecting and preparing an egg yolks by predetermined quality guidelines, then treating egg yolks by washing with water, and to remove membranes of egg yolks;
(b) hydrolyzing said treated egg yolks with a lipase enzyme preparation at 35° C. for 2 hours to create enzyme-treated egg yolks; wherein the lipase enzyme preparation has 0.05%-1.5% by weight of the said treated egg yolks;
(c) adding and homogeneously dissolving acetone solution into the enzyme-treated egg yolks for 5-10 minutes, then let stand at 3° C.-5° C. to 2-3 hours for precipitate phospholipids and proteins;
(d) creating a first precipitate by performing the steps in the following order:
(d1) filtering said phospholipid and protein precipitate generated at step (c);
(d2) washing said precipitate at step (d1) with acetone solution, and then filtering; repeating steps of at step (d2) 4 times;
(d3) removing acetone from said precipitate at step (d2) to create the first precipitate;
(e) creating a second precipitate by adding and homogeneously dissolving 5 parts of ethanol solution with 1 part of the first precipitate at 2° C.-5° C. for 5-8 minutes, and then filtering;
(f) creating a basic mixture by performing the steps in the following order:
adding and homogeneously dissolving 5 parts of ethanol 91% with 1 part of the second precipitate, and admixing a powder of activated carbon; then let stand for 15 min;
performing extraction and removing ethanol by rotary evaporator to obtain the basic mixture;
wherein the powder of activated carbon has 0.005% by total weight of ethanol 91% and the second precipitate; and
(g) creating the lecithin extract ingredients from egg yolk by performing the steps in the following order:
adding and homogeneously dissolving 3 parts of petroleum ether with 1 part of the basic mixture and 1 part of acetone, then let stand at 2° C.-5° C. for 1-3 hours;
removing the solvent layer on the surface, twice extracting by chloroform, and removing the solvent at low pressure to obtain the lecithin extract ingredients from egg yolk;
wherein (B) the lecithin extract mixture is selected from plant materials group consisting of an avocado by-product, an eggplant (Solanum melongena), a tomato seeds (Solanum lycopersicum), a chili seeds, a sesame seeds (Sesamum indicum), and a rice bran;
wherein (B) the lecithin extract mixture is obtained by performed in a specific order from (a′) to (g′) comprising:
(a′) creating a first mixture by performing the steps in the following order:
chopping/cutting 1 part of an avocado by-product and 3 parts of an eggplant (Solanum melongena) to obtain a temporary mixture;
treating the temporary mixture by steam at 100° C. for 5 minutes to obtain a steam-treated temporary mixture;
grinding steam-treated temporary mixture to obtain a temporary powder; and
drying the temporary powder having moisture content below 7% to obtain the first mixture;
(b′) creating a second mixture by performing the steps in the following order:
admixing 1 part of a tomato seeds (Solanum lycopersicum) with 1 part of a chili seeds and 2 parts of a sesame seeds (Sesamum indicum) to obtain a seeds mixture;
treating the seeds mixture by steam at 100° C. for 5 minutes, then grinding to obtain a seeds powder;
treating the seeds powder by cellulose enzyme ingredient at 28° C.-35° C. for 1 hour to obtain an enzyme-treated seeds powder; wherein cellulose-degrading enzyme ingredient has 0.05%-0.5% by weight of the seeds powder;
drying the enzyme-treated seeds powder having moisture content below 7% to obtain the second mixture;
(c′) adding into the soxhlet extractor consisting of 1 part of the first mixture with 1 part of the second mixture, 1 part of a rice bran has moisture content less than 7%, and (10-15) parts of ethyl ether solvent; all proceed to extract for 8 hours to obtain a temporary solution and a residue;
(d′) creating a first solution by centrifugation of the temporary solution;
(e′) creating a second solution by treating the residue with the lipase enzyme preparation at 35° C. for 2 hours; wherein the lipase enzyme preparation has 0.05%-1.5% by weight of the residue;
centrifuging said enzyme-treated residue and removing solvents by vacuum evaporator at 40° C. to obtain the second solution;
(f′) creating a foundation solution by adding and homogeneously dissolving the first solution with the second solution, and the powder of activated carbon, then let stand for 15 minutes, and centrifuging; wherein the powder of activated carbon has 0.005% by total weight of the first solution and the second solution; and
(g′) creating the lecithin extract mixture by performing the steps in the following order:
(g1′) adding and homogeneously dissolving cold acetone (4° C.) into the foundation solution for 5 minutes, then let stand for 3 hours at 5° C. to precipitate lecithin;
(g2′) washing said lecithin precipitate at step (g1′) by acetone at room temperature, and then filtering; repeating steps of step (g2′) 5 times;
(g3′) adding and homogeneously dissolving cold ethanol with said precipitate at step (g2′) for 8-10 minutes; and
(g4′) filtering and removing solvents by vacuum evaporator at 40° C., then twice extracting by chloroform, and removing the solvent at low pressure to obtain the lecithin extract ingredients from plants.

2. The composition of claim 1, wherein the lipase enzyme preparation obtained from a germinated dragon fruit seeds include the following steps:
(i) preparing the germinated dragon fruit seeds by following these steps in order:
selecting and preparing a dragon fruits by predetermined quality guidelines;
washing, peeling, cutting/chopping said dragon fruits into pieces and soaking with an enzyme solution according a ratio 1:5 at 30° C.-35° C. for 12 hours;
wherein the enzyme solution is obtained by homogeneously dissolving 1 part of an enzyme preparation with 1000 parts of water;
wherein the enzyme preparation including 2 parts of cellulase enzyme and 3 parts of pectinase enzyme;
wherein said cellulase enzyme is the biomass obtained by the process of increasing the biomass of microbial strains including Bacillus lichenifomis DA 15, Bacillus subtilis DA 33, and Bacillus megaterium NT09;
wherein said pectinase enzyme is the biomass obtained by the process of increasing the biomass of microbial strains including Aspergillus niger X5, and Aspergillus niger X9;
treating said enzyme-treated dragon fruits by steam at the temperature of 90° C. for 3 minutes;
scrubbing said steam-treated dragon fruits by multipurpose scrubber separating a part of pure, and a part of seeds;
soaking the part of seeds with GA3 solution (Gibberellic acid solution) at a ratio of 1:10 for 6 hours at 30° C.-35° C.; wherein GA3 solution is obtained by homogeneously dissolving 0.5 mg of GA3 with 1L of water;
incubating said GA3-treated part of seeds at 30° C.-35° C. for 8-12 hours, and maintain humidity of 75%-80% to obtain the germinated dragon fruit seeds;
(ii) creating a solution after centrifugation by following these steps in order:
grinding a mixture consisting of ½ part of water and 1 part of the germinated dragon fruit seeds;
stirring said after grinding mixture for 30 minutes;
centrifuging said after stirring mixture at 13000 rpm for 10 minutes at 4° C. to obtain a solution after centrifugation; and
(iii) freeze-drying the solution after centrifugation to obtain the lipase enzyme preparation form powder, that having activity of 12-16 TU/mg powder.

3. The composition of claim 2, wherein the dragon fruits are selected from types of dragon fruits consisting of red flesh dragon fruit (Hylocereus costaricensis), white flesh dragon fruit (Selenicereus undatus), Gele pitahaya (Hylocereus megalanthus), Purple and pink dragon fruit (Hylocereus undatus costaricensis), and combinations thereof.

4. The composition of claim 1, wherein the egg yolks are selected from eggs of various types of poultry consisting of chicken (Gallus gallus domesticus, Gallus gallus otomesticus), red-feathered wild chicken (Gallus gallus spadiceus), muskuseend (Cairina moschata forma domestica), grass goose (Cynopsis sinensis), mallard (Anas platyrhynchos), quail (Coturnix japonica), and combinations thereof.

5. The composition of claim 1, wherein the avocado by-product include skins and seeds selected from types of avocado consisting of avocado 034 (Persea americana), and avocado wax (Persea americana ‘Maluma’), and combinations thereof.

6. The composition of claim 1, wherein the chili seeds are selected from types of chili consisting of celestial chili (Celestial chili), Dangerous chili (Capsicumn annuum L.), Ba tri chili (Golden Horn Peppers), and combinations thereof.

7. A phospholipid composition applied to make the nano liquid product containing curcumin have the ability to treating burns and increasing the effect of scar healing, wherein the nano liquid product containing curcumin obtained by homogenously mixing a curcumin nano ingredient with the phospholipid composition in a ratio of 1:1 for 4 hours at 120° C. by emulsifying equipment;
wherein the curcumin nano ingredient is obtained by performed in a specific order from (i) to (v) comprising:
(i) preparing a dispersed phase by dissolving 4 parts of a curcumin with 5 parts of ethanol;
(ii) creating a carrier mixture by homogeneous dissolving 1.5 parts PEG (polyethylene glycol) with 6 parts EG (ethylene glycol) and 2 parts water, with the combination of ultrasonic vibration for 2 hours at room temperature;
(iii) creating a homogeneous mixture by homogeneous dissolving 1.6 parts of the dispersed phase at step (i) with 1.5 parts of the carrier mixture at step (ii), and 2 parts of the phospholipid composition by emulsifying equipment;
(iv) keeping the homogeneous mixture overnight; and
(v) centrifuging said homogenized mixture at step (iv) with a speed of 5000 rpm for 10 min, and repeated 6 times of centrifugation to obtain the curcumin nano ingredient form microemulsion;
wherein the phospholipid composition obtained from the process of forming a homogeneous mixture by mixing (A) a lecithin extract ingredients from egg yolk with (B) a lecithin extract ingredients from plants in a predetermined ratio of (1-3): (1-3); and
then adjusting moisture of the homogeneous mixture below 10%;
(A) the lecithin extract ingredients from egg yolk is obtained by performed in a specific order from (a) to (g) comprising:
(a) selecting and preparing an egg yolks by predetermined quality guidelines, then treating egg yolks by washed with water, and using the sieve to remove membranes of egg yolks;
(b) hydrolyzing said treated egg yolks with a lipase enzyme preparation at 35° C. for 2 hours to create an enzyme-treated egg yolks; wherein the lipase enzyme preparation having 0.05%-1.5% by weight of the said treated egg yolks;
(c) adding and homogeneously dissolving acetone solution to the enzyme-treated egg yolks for 5-10 minutes, then let stand at 3° C.-5° C. for 2-3 hours for precipitate phospholipids and proteins;
(d) creating a first precipitate by performing the steps in the following order:
(d1) filtering said phospholipid and protein precipitate generated at step (c);
(d2) washing said precipitate at step (d1) with acetone solution, and then filtering;
repeating steps of at step (d2) 4 times;
(d3) removing acetone from said precipitate at step (d2) to create the first precipitate;
(e) creating a second precipitate by adding and homogeneously dissolving 5 parts of ethanol solution with 1 part of the first precipitate at 2° C.-5° C. for 5-8 minutes, and then filtering;
(f) creating a basic mixture by performing the steps in the following order:
adding and homogeneously dissolving 5 parts of ethanol 91% with 1 part of the second precipitate, and admixing a powder of activated carbon; then let stand for 15 min;
performing extraction and removing ethanol by rotary evaporator to obtain the basic mixture;
wherein the powder of activated carbon having 0.005% by total weight of ethanol 91% and the second precipitate; and
(g) creating the lecithin extract ingredients from egg yolk by performing the steps in the following order:
adding and homogeneously dissolving 3 parts of petroleum ether with 1 part of the basic mixture and 1 part of acetone, then let stand at 2° C.-5° C. for 1-3 hours;
removing the solvent layer on the surface, twice extracting by chloroform, and removing the solvent at low pressure to obtain the lecithin extract ingredients from egg yolk;
(B) the lecithin extract ingredients from plants is obtained by performed in a specific order from (a′) to (g′) comprising:
(a′) creating a first mixture by performing the steps in the following order:
chopping/cutting 1 part of an avocado by-product and 3 parts of an eggplant (Solanum melongena) to obtain a temporary mixture;
treating the temporary mixture by steam at 100° C. for 5 minutes to obtain a steam-treated temporary mixture;
grinding steam-treated temporary mixture to obtain a temporary powder; and
drying the temporary powder having moisture content below 7% to obtain the first mixture;
(b′) creating a second mixture by performing the steps in the following order:
admixing 1 part of a tomato seeds (Solanum lycopersicum) with 1 part of a chili seeds and 2 parts of a sesame seeds (Sesamum indicum) to obtain a seeds mixture;
treating the seeds mixture by steam at 100° C. for 5 minutes, then grinding to obtain a seeds powder;
treating the seeds powder by cellulose enzyme ingredient at 28° C.-35° C. for 1 hour to obtain an enzyme-treated seeds powder; wherein cellulose enzyme ingredient having 0.05%-0.5% by weight of the seeds powder;
drying the enzyme-treated seeds powder having moisture content below 7% to obtain the second mixture;
(c′) adding into the soxhlet extractor consisting of 1 part of the first mixture with 1 part of the second mixture, 1 part of a rice bran having moisture content less than 7%, and (10-15) parts of ethyl ether solvent; all proceed to extract for 8 hours to obtain a temporary solution and a residue;
(d′) creating a first solution by centrifugation of the temporary solution;
(e′) creating a second solution by treated the residue with the lipase enzyme preparation at 35° C. for 2 hours; wherein the lipase enzyme preparation having 0.05%-1.5% by weight of the residue; centrifuging said enzyme-treated residue and removing solvents by vacuum evaporator at 40° C. to obtain the second solution;
(f′) creating a foundation solution by adding and homogeneously dissolving the first solution with the second solution, and the powder of activated carbon, then let stand for 15 minutes, and centrifuging; wherein the powder of activated carbon having 0.005% by total weight of the first solution and the second solution; and
(g′) creating the lecithin extract ingredients from plants by performing the steps in the following order:
(g1′) adding and homogeneously dissolving cold acetone (4° C.) to the foundation solution for 5 minutes, then let stand for 3 hours at 5° C. for precipitate lecithin;
(g2′) washing said lecithin precipitate at step (g1′) by acetone at room temperature, and then filtering; repeating steps of step (g2′) 5 times;
(g3′) adding and homogeneously dissolving cold ethanol with said precipitate at step (g2′) for 8-10 minutes; and
(g4′) filtering and removing solvents by vacuum evaporator at 40° C., then twice extracting by chloroform, and removing the solvent at low pressure to obtain the lecithin extract ingredients from plants;
wherein the product is used at a dose of 0.05-0.1 mL/cm2 of skin, with a frequency of twice daily to reduce the area of the burns, and increasing the concentration of hydroxyproline in the skin.

8. The composition of claim 7, wherein the lipase enzyme preparation obtained from a germinated dragon fruit seeds include the following steps:
(i) preparing the germinated dragon fruit seeds by following these steps in order:
selecting and preparing a dragon fruits by predetermined quality guidelines;
washing, peeling, cutting/chopping said dragon fruits into pieces and soaking with an enzyme solution according a ratio 1:5 at 30° C.-35° C. for 12 hours;
wherein the enzyme solution is obtained by homogeneously dissolving 1 part of an enzyme preparation with 1000 parts of water;
wherein the enzyme preparation including 2 parts of cellulase enzyme and 3 parts of pectinase enzyme;
wherein cellulase enzyme is the biomass obtained by the process of increasing the biomass of microbial strains including Bacillus lichenifomis DA 15, Bacillus subtilis DA 33, and Bacillus megaterium NT09;
wherein pectinase enzyme is the biomass obtained by the process of increasing the biomass of microbial strains including Aspergillus niger X5, and Aspergillus niger X9;
treating said enzyme-treated dragon fruits by steam at the temperature of 90° C. for 3 minutes;
scrubbing said steam-treated dragon fruits by multipurpose scrubber separating a part of pure, and a part of seeds;
soaking the part of seeds with GA3 solution at a ratio of 1:10 for 6 hours at 30° C. 35° C.; wherein GA3 solution is obtained by homogeneously dissolving 0.5 mg of GA3 with 1L of water;
incubating said GA3-treated part of seeds at 30° C.-35° C. for 8-12 hours, and maintain humidity of 75%-80% to obtain the germinated dragon fruit seeds;
(ii) creating a solution after centrifugation by following these steps in order:
grinding a mixture consisting of ½ part of water and 1 part of the germinated dragon fruit seeds;
stirring said after grinding mixture for 30 minutes;
centrifuging said after stirring mixture at 13000 rpm for 10 minutes at 4° C. to obtain a solution after centrifugation; and
(iii) freeze-drying the solution after centrifugation to obtain the lipase enzyme preparation form powder, that having activity of 12-16 TU/mg powder.

9. The composition of claim 8, wherein the dragon fruits are selected from types of dragon fruits including red flesh dragon fruit (Hylocereus costaricensis), white flesh dragon fruit (Selenicereus undatus), Gele pitahaya (Hylocereus megalanthus), and Purple and pink dragon fruit (Hylocereus undatus costaricensis).

10. The composition of claim 7, wherein the egg yolks are selected from eggs of various types of poultry including chicken (Gallus gallus domesticus, Gallus gallus otomesticus), red-feathered wild chicken (Gallus gallus spadiceus), muskuseend (Cairina moschata forma domestica), grass goose (Cynopsis sinensis), mallard (Anas platyrhynchos), and quail (Coturnix japonica).

11. The composition of claim 7, wherein the avocado by-product include skins and seeds selected from types of avocado including avocado 034 (Persea americana), and avocado wax (Persea americana ‘Maluma’).

12. The composition of claim 7, wherein the chili seeds are selected from types of chili including celestial chili (Celestial chili), Dangerous chili (Capsicumn annuum L.), and Ba tri chili (Golden Horn Peppers).

13. A phospholipid composition applied to make the nano liquid product containing curcumin have the ability to treating burns and increasing the effect of scar healing, wherein the nano liquid product containing curcumin obtained by homogenously mixing a curcumin nano ingredient with a foundation mixture in a ratio of 1:1 for 4 hours at 120° C. by emulsifying equipment;
wherein the curcumin nano ingredient is obtained by performed in a specific order from (i) to (v) comprising:
(i) preparing a dispersed phase by dissolving 4 parts of a curcumin with 5 parts of ethanol;
(ii) creating a carrier mixture by homogeneous dissolving 1.5 parts PEG (polyethylene glycol) with 6 parts EG (ethylene glycol) and 2 parts water, with the combination of ultrasonic vibration for 2 hours at room temperature;
(iii) creating a homogeneous mixture by homogeneous dissolving 1.6 parts of the dispersed phase at step (i) with 1.5 parts of the carrier mixture at step (ii), and 2 parts of the phospholipid composition by emulsifying equipment;
(iv) keeping the homogeneous mixture overnight; and
(v) centrifuging said homogenized mixture at step (iv) with a speed of 5000 rpm for 10 min, and repeated 6 times of centrifugation to obtain the curcumin nano ingredient form microemulsion;
wherein the foundation mixture comprising cholesterol, folic acid, nanocurumin, tocopherol, xanthan gum, and the phospholipid composition;
wherein the phospholipid composition obtained from the process of forming a homogeneous mixture by mixing (A) a lecithin extract ingredients from egg yolk with (B) a lecithin extract ingredients from plants in a predetermined ratio of (1-3): (1-3); and
then adjusting moisture of the homogeneous mixture below 10%;
(A) the lecithin extract ingredients from egg yolk is obtained by performed in a specific order from (a) to (g) comprising:
(a) selecting and preparing an egg yolks by predetermined quality guidelines, then treating egg yolks by washed with water, and using the sieve to remove membranes of egg yolks;
(b) hydrolyzing said treated egg yolks with a lipase enzyme preparation at 35° C. for 2 hours to create an enzyme-treated egg yolks; wherein the lipase enzyme preparation having 0.05%-1.5% by weight of the said treated egg yolks;
(c) adding and homogeneously dissolving acetone solution to the enzyme-treated egg yolks for 5-10 minutes, then let stand at 3° C.-5° C. for 2-3 hours for precipitate phospholipids and proteins;
(d) creating a first precipitate by performing the steps in the following order:
(d1) filtering said phospholipid and protein precipitate generated at step (c);
(d2) washing said precipitate at step (d1) with acetone solution, and then filtering; repeating steps of at step (d2) 4 times;
(d3) removing acetone from said precipitate at step (d2) to create the first precipitate;
(e) creating a second precipitate by adding and homogeneously dissolving 5 parts of ethanol solution with 1 part of the first precipitate at 2° C.-5° C. for 5-8 minutes, and then filtering;
(f) creating a basic mixture by performing the steps in the following order:
adding and homogeneously dissolving 5 parts of ethanol 91% with 1 part of the second precipitate, and admixing a powder of activated carbon; then let stand for 15 min;
performing extraction and removing ethanol by rotary evaporator to obtain the basic mixture;
wherein the powder of activated carbon having 0.005% by total weight of ethanol 91% and the second precipitate; and
(g) creating the lecithin extract ingredients from egg yolk by performing the steps in the following order:
adding and homogeneously dissolving 3 parts of petroleum ether with 1 part of the basic mixture and 1 part of acetone, then let stand at 2° C.-5° C. for 1-3 hours;
removing the solvent layer on the surface, twice extracting by chloroform, and removing the solvent at low pressure to obtain the lecithin extract ingredients from egg yolk;
(B) the lecithin extract ingredients from plants is obtained by performed in a specific order from (a′) to (g′) comprising:
(a′) creating a first mixture by performing the steps in the following order:
chopping/cutting 1 part of an avocado by-product and 3 parts of an eggplant (Solanum melongena) to obtain a temporary mixture;
treating the temporary mixture by steam at 100° C. for 5 minutes to obtain a steam-treated temporary mixture;
grinding steam-treated temporary mixture to obtain a temporary powder; and
drying the temporary powder having moisture content below 7% to obtain the first mixture;
(b′) creating a second mixture by performing the steps in the following order:
admixing 1 part of a tomato seeds (Solanum lycopersicum) with 1 part of a chili seeds and 2 parts of a sesame seeds (Sesamum indicum) to obtain a seeds mixture;
treating the seeds mixture by steam at 100° C. for 5 minutes, then grinding to obtain a seeds powder;
treating the seeds powder by cellulose enzyme ingredient at 28° C.-35° C. for 1 hour to obtain an enzyme-treated seeds powder; wherein cellulose enzyme ingredient having 0.05%-0.5% by weight of the seeds powder;
drying the enzyme-treated seeds powder having moisture content below 7% to obtain the second mixture;
(c″) adding into the soxhlet extractor consisting of 1 part of the first mixture with 1 part of the second mixture, 1 part of a rice bran having moisture content less than 7%, and (10-15) parts of ethyl ether solvent; all proceed to extract for 8 hours to obtain a temporary solution and a residue;
(d′) creating a first solution by centrifugation of the temporary solution;
(e′) creating a second solution by treated the residue with the lipase enzyme preparation at 35° C. for 2 hours; wherein the lipase enzyme preparation having 0.05%-1.5% by weight of the residue; centrifuging said enzyme-treated residue and removing solvents by vacuum evaporator at 40° C. to obtain the second solution;
(f′) creating a foundation solution by adding and homogeneously dissolving the first solution with the second solution, and the powder of activated carbon, then let stand for 15 minutes, and centrifuging; wherein the powder of activated carbon having 0.005% by total weight of the first solution and the second solution; and
(g′) creating the lecithin extract ingredients from plants by performing the steps in the following order:
(g1′) adding and homogeneously dissolving cold acetone (4° C.) to the foundation solution for 5 minutes, then let stand for 3 hours at 5° C. for precipitate lecithin;
(g2′) washing said lecithin precipitate at step (g1′) by acetone at room temperature, and then filtering; repeating steps of step (g2′) 5 times;
(g3′) adding and homogeneously dissolving cold ethanol with said precipitate at step (g2′) for 8-10 minutes; and
(g4′) filtering and removing solvents by vacuum evaporator at 40° C., then twice extracting by chloroform, and removing the solvent at low pressure to obtain the lecithin extract ingredients from plants;
wherein the product is used at a dose of 0.05-0.1 mL/cm2 of skin, with a frequency of twice daily to reduce the area of the burns, and increasing the concentration of hydroxyproline in the skin.

14. The composition of claim 13, wherein the lipase enzyme preparation obtained from a germinated dragon fruit seeds include the following steps:
(i) preparing the germinated dragon fruit seeds by following these steps in order:
selecting and preparing a dragon fruits by predetermined quality guidelines;
washing, peeling, cutting/chopping said dragon fruits into pieces and soaking with an enzyme solution according a ratio 1:5 at 30° C.-35° C. for 12 hours;
wherein the enzyme solution is obtained by homogeneously dissolving 1 part of an enzyme preparation with 1000 parts of water;
wherein the enzyme preparation including 2 parts of cellulase enzyme and 3 parts of pectinase enzyme;
wherein cellulase enzyme is the biomass obtained by the process of increasing the biomass of microbial strains including Bacillus lichenifomis DA 15, Bacillus subtilis DA 33, and Bacillus megaterium NT09;
wherein pectinase enzyme is the biomass obtained by the process of increasing the biomass of microbial strains including Aspergillus niger X5, and Aspergillus niger X9;
treating said enzyme-treated dragon fruits by steam at the temperature of 90° C. for 3 minutes;
scrubbing said steam-treated dragon fruits by multipurpose scrubber separating a part of pure, and a part of seeds;
soaking the part of seeds with GA3 solution at a ratio of 1:10 for 6 hours at 30° C. 35° C.; wherein GA3 solution is obtained by homogeneously dissolving 0.5 mg of GA3 with 1L of water;
incubating said GA3-treated part of seeds at 30° C.-35° C. for 8-12 hours, and maintain humidity of 75%-80% to obtain the germinated dragon fruit seeds;
(ii) creating a solution after centrifugation following these steps in order:
grinding a mixture consisting of ½ part of water and 1 part of the germinated dragon fruit seeds;
stirring said after grinding mixture for 30 minutes;
centrifuging said after stirring mixture at 13000 rpm for 10 minutes at 4° C. to obtain a solution after centrifugation; and
(iii) freeze-drying the solution after centrifugation to obtain the lipase enzyme preparation form powder, that having activity of 12-16 TU/mg powder.

15. The composition of claim 14, wherein the dragon fruits are selected from types of dragon fruits including red flesh dragon fruit (Hylocereus costaricensis), white flesh dragon fruit (Selenicereus undatus), Gele pitahaya (Hylocereus megalanthus), and Purple and pink dragon fruit (Hylocereus undatus costaricensis).

16. The composition of claim 13, wherein the egg yolks are selected from eggs of various types of poultry including chicken (Gallus gallus domesticus, Gallus gallus otomesticus), red-feathered wild chicken (Gallus gallus spadiceus), muskuseend (Cairina moschata forma domestica), grass goose (Cynopsis sinensis), mallard (Anas platyrhynchos), and quail (Coturnix japonica).

17. The composition of claim 13, wherein the avocado by-product include skins and seeds selected from types of avocado including avocado 034 (Persea americana), and avocado wax (Persea americana ‘Maluma’).

18. The composition of claim 13, wherein the chili seeds are selected from types of chili including celestial chili (Celestial chili), Dangerous chili (Capsicumn annuum L.), and Ba tri chili (Golden Horn Peppers).

19. The composition of claim 13, wherein the foundation mixture comprising:
cholesterol having 18%-25% by weight;
folic acid having 8%-12% by weight;
nanocurumin having 12%-20% by weight;
tocopherol having 2%-5% by weight;
xanthan gum having 2%-5% by weight;
Camellia Sinensis extracts ingredient having 2%-5% by weight;
aloe vera extracts ingredient having 0.25%-1% by weight; and
the remainder is the phospholipid composition.

20. The composition of claim 13, wherein the foundation mixture comprising:
cholesterol having 21% by weight;
folic acid having 9.5% by weight;
nanocurumin having 15% by weight;
tocopherol having 3% by weight;
xanthan gum having 3% by weight;
Camellia Sinensis extracts ingredient having 3% by weight;
aloe vera extracts ingredient having 0.5% by weight; and
the remainder is the phospholipid composition.
